(19) United States
(12) Patent Application Publication
(10) Pub. No.: US 2025/0228757 A1
(43) Pub. Date: Jul. 17, 2025

(54) SUNSCREEN COMPOUNDS AND FORMULATIONS

(71) Applicant: Oregon State University, Corvallis, OR (US)

(72) Inventors: Jan Frederik Stevens, Corvallis, OR (US); Gitali Indra, Corvallis, OR (US); Arup K. Indra, Corvallis, OR (US); Evan L. Carpenter, Emeryville, CA (US)

(73) Assignee: Oregon State University, Corvallis, OR (US)

(21) Appl. No.: 19/015,263

(22) Filed: Jan. 9, 2025

Related U.S. Application Data
(60) Provisional application No. 63/619,866, filed on Jan. 11, 2024.

(51) Int. Cl. A61K 8/42; A61Q 17/04; C07C 233/33

(52) U.S. Cl. CPC A61K 8/42; A61Q 17/04; C07C 233/33; A61K 2800/5922; A61K 2800/81

(57) ABSTRACT

Amide lipid conjugates of benzyl isothiocyanates are disclosed. The benzyl isothiocyanate is conjugated to a fatty acid, such as a C₁₂-C₂₆ fatty acid. The disclosed amide lipid conjugates have a structure according to Formula I, where one of R^{1A} and R^{1B} is alkoxy or hydroxy, and the other of R^{1A} and R^{1B} is —H; and R² is C₁₂-C₂₆ aliphatic. Sunscreen formulations include a compound according to Formula I and one or more excipients. The sunscreen formulation may further include one or more additional sunscreen active agents.

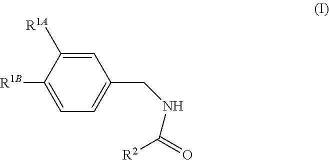

### CROSS-REFERENCE TO RELATED APPLICATION

This application claims the benefit of the earlier filing date of U.S. Provisional Application No. 63/619,866, filed Jan. 11, 2024, which is incorporated by reference in its entirety herein.

### ACKNOWLEDGEMENT OF GOVERNMENT SUPPORT

This invention was made with government support under award No. 1R01ES016629, awarded by the National Institutes of Health, and award No. T32AT010131, awarded by the National Institutes of Health. The government has certain rights in the invention.

### BACKGROUND

Chronic overexposure of the skin to solar ultraviolet radiation (UVR) is a common dermatological concern that, without proper mitigation, causes cumulative damage leading to photoaging and increased risk of cancer. Accordingly, skin cancers are the most frequently diagnosed form of cancer, and the burden of melanoma, the deadliest skin disease, is expected to increase globally over the next couple of decades to 2040 (Arnold et al., JAMA Dermatol 2022, 158:495-503). UVR is known to be the primary initiator of melanomagenesis in at least three-quarters of cases where key driver mutations in melanocyte proto-oncogenes, like cell proliferation/survival regulators BRAF and NRAS, arise and initiate tumorigenesis (Arnold et al., Int J Cancer 2018, 143:1305-1324; Schadendorf et al., Lancet 2018, 392 (971-094). Therefore, photoprotection of the skin is essential for combating the deleterious aspects of UVR. Solar UVR is categorized into three wavelengths UVA (320-400 nm), UVB (290-320 nm), and UVC (200-280 nm). The earth's ozone layer absorbs UVC wavelengths, preventing them from passing through the atmosphere, whereas UVA and UVB wavelengths can be incident upon the skin (Shin, Anim Cells Syst (Seoul) 2020, 24:181-188). UVB is the more directly damaging of these two wavelengths and is mainly responsible for UV-induced sunburns and erythema Meyer et al., Curr Probl Dermatol 2021, 55:53-61). One molecular mechanism by which UVB injures the skin is direct DNA damage induced in skin cells, resulting in mutagenic cyclobutane pyrimidine dimers (CPDs) and 6,4-photoproducts (DeHaven et al., J Cosmet Dermatol 2014, 13:99-102). For this reason, sunscreens are formulated to ensure protection against UVB wavelengths, and this photoprotective potency is indicated by the sunscreen's sun protection factor (SPF) (Wang et al., J Am Acad Dermatol 2017, 77:42-47).

The active ingredients in commercial sunscreens include inorganic and organic compounds that act in differing ways to mitigate UVR. Inorganic active ingredients consist of titanium dioxide and zinc oxide, which act as UV filters by scattering and reflecting UV light. Organic active ingredients include polyphenols and flavonoids that absorb UV light or have antioxidant properties (Guan et al., Am J Clin Dermatol 2021, 22:819-828). UV absorption is due to the presence of an aromatic ring conjugated to a carbonyl group which is a structure capable of absorbing UV, entering an excited state, and then emitting that energy as heat (Gabros et al., In StatPearls, Treasure Island (FL) 2023).

Recently, there have been some controversies regarding the deleterious side effects of commercial sunscreen ingredients for both human health and marine ecosystems. Toxicology studies suggest that the additive oxybenzone, given a high enough dose, can have photoallergenic and estrogenic effects (Mancuso et al., Am J Clin Dermatol 2017, 18:643-650; Mustieles et al., Environ Int 2023, 173:107739). Ecological studies have indicated that inorganic and organic additives could exhibit unfavorable environmental toxicology that negatively impacts marine organisms like corals, algae, arthropods, and fish (Chatzgianni et al., Ecotoxicology 2022, 31:1331-1345). These concerns highlight the need for novel sunscreen additives that avoid these side effects.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Schematic representation of the synthesis of N-(3-methoxybenzyl)eicos-5-enamide (MBamide or MBA) from 3-methoxybenzyl isothiocyanate (MBITC) and the meadowfoam fatty acid, Z-5-eicosenoic acid.

FIGS. 2A-2B, Time-of-Flight (ToF) mass spectroscopy of (Z)—N-(3-methoxybenzyl)eicos-5-enamide (FIG. 2A) and a quadrupole-ToF (Q-ToF) MS/MS spectrum of the molecular ion (FIG. 2B).

FIG. 3. 1H NMR Spectrum of (Z)—N-(3-methoxybenzyl)eicos-5-enamide (MBamide) in methanol-d4 (700 MHz).

FIGS. 4A-4B. UV spectra of (Z)—N-(3-methoxybenzyl)eicos-5-enamide (MBamide) (FIG. 4A) and 3-methoxybenzyl isothiocyanate (MBITC) (FIG. 4B).

FIGS. 5A-5B. Lipid conjugation of MBITC reduces cytotoxicity in human keratinocyte cultures. (FIG. 5A) Dose-response MTS assay for survival of HaCaT cells incubated with MBITC or MBA for 48 h. (FIG. 5B) Dose-response CellTiter-Glo® assay (Promega Corporation, Madison, WI) for survival of human primary epidermal keratinocytes treated with MBA for 48 h. Data represent mean±SD from two independent experiments. Significance determined by one-way ANOVA. *=p<0.05, **=p<0.01, ***=p<0.001, ****=p<0.0001

FIGS. 6A-6C. MBITC and MBA absorption of UVB and photoprotection assays. UVB absorption assay for (FIG. 6A) MBITC or (FIG. 6B) MBA measuring the relative time needed to reach a specified dose of 5 or 10 mJ/cm². (FIG. 6C) HaCaT cells were treated with ethanol (EtOH), MBITC, or MBA for 12 h, irradiated with 10 mJ/cm² UVB, and then an MTS survival assay was performed after 36 h. Data represent mean±SD from two independent experiments. Significance determined by one-way ANOVA. *=p<0.05, **=p<0.01, ***=p<0.001, ****=p<0.0001

FIGS. 7A-7E. MBA prevents UVB-induced DNA damage. (FIG. 7A) Immunocytochemistry for cyclobutane pyrimidine dimers (CPDs) in human primary keratinocytes treated with EtOH or indicated doses of MBA for 12 h, irradiated with 5 mJ/cm² UVB, and then fixed after 36 h. Bar indicates 50 μm. Quantification of immunocytochemistry for (FIG. 7B) CPD+ cells or (FIG. 7C) CPD signal intensity via corrected total cellular fluorescence (CTCF). (FIG. 7D) Immunoblotting and (FIG. 7E) quantification for DNA double-strand break marker p-γH2A.X in human primary keratinocytes treated with MBITC or MBA for 12 h, irradiated with 5 mJ/cm² UVB, and harvested after 36 h. Data represent mean±SD from two independent experiments performed in duplicate. Significance was determined by one sample t-test (C, E). ns=nonsignificant, *=p<0.05

FIGS. 8A-8B. MBA photoprotection assay in human primary keratinocytes. Cell count assays for (FIG. 8A) dose-response or (FIG. 8B) time kinetics of human primary epidermal keratinocytes treated with indicated doses of MBA for 12 h, irradiated with 10 mJ/cm² UVB, and CyQuant® assay (ThermoFisher Scientific, Waltham, MA) performed.

### DETAILED DESCRIPTION

Natural products offer a promising reservoir of organic sunscreen compounds that might overcome the potential issues with current additives. Prospective sunscreen phytochemicals include flavonoids, polyphenols, lignin, and polysaccharides due to their UV absorptive and antioxidant properties (Li et al., Animal Model Exp Ed 2023, 6:183-195). For example, the ethanolic extract of rambutan (Nephelium lappaceum L) contains ellagic acid, corilagin, and geraniin polyphenols that act to absorb UVB wavelengths (Mota et al., J Photochem Photobiol B 2020, 205:111837).

Derivatives of the singular meadowfoam (Limnanthes alba) glucosinolate, glucolimnanthin, exhibit UV photoprotective and anti-aging properties in human skin equivalents (Carpenter et al., Front Pharmacol 2018, 9:477). For example, 3-methoxybenzyl isothiocyanate (MBITC) and 3-methoxybenzyl acetonitrile (MPACN), were found to reduce UVB-induced DNA damage, proliferation, and matrix-metalloproteinase expression. This disclosure concerns novel amide lipid conjugates of benzyl isothiocyanates, such as alkoxybenzyl or hydroxybenzyl isothiocyanates. In some aspects, the lipid is derived from fatty acids found in meadowfoam (Limnanthes alba). In certain aspects, the disclosed compounds exhibited less cytotoxicity than 3-methoxybenzyl isothiocyanate (MBITC) while still retaining the photoprotective properties of MBITC by absorbing UVB light and reducing UV-induced DNA damage. Overall, lipid conjugation of a UVB-absorbing compound yielded an improved preliminary safety profile at the cost of absorptive capacity.

### I. Definitions and Abbreviations

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0).

In order to facilitate review of the various aspects of the disclosure, the following explanations of specific terms are provided:

Aliphatic: A substantially hydrocarbon-based compound, or a radical thereof (e.g., C₆H₁₃, for a hexane radical), including alkanes, alkenes, and alkynes, including cyclic versions thereof, and further including straight- and branched-chain arrangements, and all stereo and position isomers as well. Unless expressly referred to as an “unsubstituted aliphatic,” an aliphatic group can either be unsubstituted or substituted. An aliphatic group can be substituted with one or more substituents (up to two substituents for each methylene carbon in an aliphatic chain, or up to one substituent for each carbon of a C═C double bond in an aliphatic chain, or up to one substituent for a carbon of a terminal methine group). Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality.

Alkoxy: A radical (or substituent) having the structure —OR, where R is a substituted or unsubstituted alkyl. Methoxy (—OCH₃) is a non-limiting exemplary alkoxy group. In a substituted alkoxy, R is alkyl substituted with a non-interfering substituent.

Cream: As used herein, the term “cream” refers to a semisolid formulation comprising an emulsion that is not pourable at room temperature (e.g., 20-25° C.). A cream typically includes >20 wt % water and volatiles, and/or <50 wt % hydrocarbons, waxes, or polyols.

Emulsion: A stable mixture of two or more immiscible substances wherein one substance (i.e., the disperse phase or minor component) is dispersed within the other (i.e., the continuous phase or major component).

Excipient: A physiologically inert substance that is used as an additive in a pharmaceutical composition. An excipient can be used, for example, to dilute an active agent and/or to modify properties of a pharmaceutical composition.

Gel: As used herein, the term “gel” refers to a semisolid formulation comprising a colloidal dispersion and containing a gelling agent. A gel typically includes >50 wt % water and volatiles.

Lotion: As used herein, the term “lotion” refers to a formulation comprising an emulsion that is pourable at room temperature (e.g., 20-25° C.). Lotions may comprise an oil-in-water or a water-in-oil emulsion.

Ointment: As used herein, the term “ointment” refers to a semisolid hydrocarbon-based formulation. An ointment typically includes >50 wt % solid and/or liquid hydrocarbons such as oils, fats, and/or waxes.

### II. Compounds and Sunscreen Formulations

Amide lipid conjugates of benzyl isothiocyanates are disclosed. In some aspects, the compounds have a structure according to Formula I

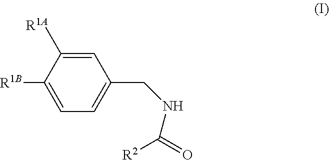

wherein one of R^{1A} and R^{1B} is alkoxy or hydroxy, and the other of R^{1A} and R^{1B} is —H; and R² is C₁₂-C₂₆ aliphatic.

In some implementations, one of R^{1A} and R^{1B} is C₁-C₃ alkoxy, e.g., methoxy, ethoxy, propoxy, or isopropoxy. In certain implementations, R^{1A} is C₁-C₃ alkoxy and R^{1B} is —H. In one implementation, R^{1A} is methoxy and R^{1B} is —H In an independent implementation, R^{1A} is —H and R^{1B} is hydroxy.

In any of the foregoing or following aspects, R² may be unsubstituted C₁₂-C₂₆ aliphatic. R² may be unsaturated or saturated. In some implementations, R² is derived from a fatty acid found in meadowfoam. Major fatty acids in meadowfoam include eicosenoic acid (C20: 1), docosenoic acid (C22: 1), and docosadienoic acid (C22: 2). In one aspect, R² is monounsaturated. In an independent aspect, R² is polyunsaturated. In another independent aspect, R² is saturated. In certain aspects, R² is unsubstituted C₁₂-C₂₆ alkenyl with one or more double bonds, such as unsubstituted C₁₈-C₂₂ alkenyl with one or two double bonds. In some implementations, the double bond has a cis or zusammen (abbreviated as “Z”) configuration in which the two groups attached to the —CH═CH— moiety are on the same side of the double bond. In some examples, R² is —(CH₂)₃CH=CH(CH₂)₁₃CH₃. In certain examples, R² is

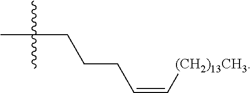

One exemplary compound according to Formula I is (Z)—N-(3-methoxybenzyl)eicos-5-enamide:

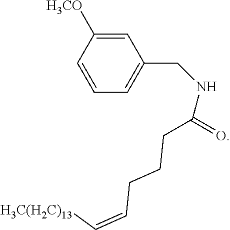

Another exemplary compound according to Formula I is (Z)—N-(4-hydroxybenzyl)eicos-5-enamide:

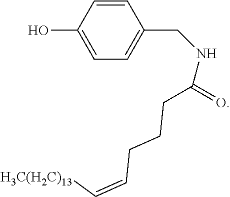

Disclosed aspects of a sunscreen formulation include a compound according to Formula I and one or more excipients. In some implementations, the formulation includes two or more compounds according to Formula I and one or more excipients. In some aspects, the formulation further includes one or more additional sunscreen active agents. In such aspects, the formulation includes one or more compounds according to Formula I, one or more additional sunscreen active agents, and one or more excipients. The formulation may be a solid, a cream, a lotion, a gel, an ointment, an oil, or a spray formulation.

In any of the foregoing or following aspects, the sunscreen formulation may comprise from 0.01 wt % to 15 wt % of the compound according to Formula I. When the sunscreen formulation includes two or more compounds according to Formula I, a total content of the compounds according to Formula I may be from 0.05 wt % to 15 wt %. In some aspects, the sunscreen formulation comprises an amount the compound or compounds according to Formula I within a range having endpoints selected from 0.01 wt %, 0.05 wt %, 0.1 wt %, 0.3 wt %, 0.5 wt %, 0.7 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, or 15 wt %. In certain aspects, the sunscreen formulation comprises 0.5 wt % to 15 wt % of the compound(s), such as from 1 wt % to 10 wt % of the compound(s). In any of the foregoing or following aspects, the compound may be (Z)—N-(3-methoxybenzyl)eicos-5-enamide, (Z)—N-(4-hydroxybenzyl)eicos-5-enamide, or a combination thereof.

Suitable excipients include, but are not limited to, plant/seed extracts, plant/seed oils, emollients, humectants, film-forming agents, thickeners (e.g., oil gelling agents), emulsifiers, diluents (e.g., water, alcohol, glycerin), fragrances, or any combination thereof. Additional excipients known to those of ordinary skill in the art of cosmetic preparations and/or sunscreen formulations also may be included. In some aspects, the one or more excipients comprise a plant or seed extract, a plant or seed oil, an alcohol (an alcohol may function as an emollient, a thickener, an emulsifier, and/or a diluent), an alkyl benzoate (an emollient), an oil gelling agent (a thickener), or any combination thereof. Exemplary plant or seed extracts or oils include, but are not limited to, Raphanus spp. plant or seed extract or oil, Sinapis spp. plant or seed extract or oil, or a combination thereof. In some examples, the plant or seed extract or oil is obtained from Raphanus sativus (radish). In some examples, the plant or seed extract or oil is obtained from Sinapis alba (white mustard). Exemplary oil gelling agents include dibutyl lauroyl glutamide, dibutyl ethylhexanoyl glutamide, octyldodecanol, dextrin fatty acid esters (e.g., dextrin palmitate), silica, and hydrogenated styrene/methylstyrene/indene copolymers, among others. Exemplary alcohols include long-chain or fatty alcohols (e.g., C₁₂-C₂₆ alcohols), such as octyldodecanol, cetyl alcohol, stearyl alcohol, and cetearyl alcohol.

In some aspects, the excipients comprise Raphanus sativus seed extract, Sinapis spp. seed extract, Limnanthes alba (meadowfoam) seed oil, bisabolol, C₁₂-C₁₅ alkyl benzoate, octyldodecanol, dibutyl lauroyl glutamide, dibutyl ethylhexanoyl glutamide, and combinations thereof. Depending on the formulation, the excipients may further include additional diluents (e.g., water, alcohol, glycerin). In one implementation, the excipients comprise, consist essentially of, or consist of a combination of Raphanus sativus seed extract and/or Sinapis spp. seed extract, Limnanthes alba (meadowfoam) seed oil, bisabolol, C₁₂-C₁₅ alkyl benzoate, octyldodecanol, dibutyl lauroyl glutamide, and dibutyl ethylhexanoyl glutamide, and, optionally, a diluent. The phrase “consist essentially of” in this context means that the sunscreen formulation does not include another excipient in an amount greater than 1 wt %, based on a total mass of the sunscreen formulation. In one representative example, the excipients comprise, consist essentially of, or consist of 17 wt % to 26 wt % Raphanus sativus seed extract and/or Sinapis spp. seed extract, 14 wt % to 18 wt % Limnanthes alba (meadowfoam) seed oil, 0.5 wt % to 1.5 wt % bisabolol, 4 wt % to 6 wt % C₁₂-C₁₅ alkyl benzoate, 18 wt % to 22 wt % octyldodecanol, 2 wt % to 3 wt % dibutyl lauroyl glutamide, and 1 wt % to 2 wt % dibutyl ethylhexanoyl glutamide, where the wt % values are based on a total mass of the sunscreen formulation.

In any of the foregoing or following aspects, the sunscreen formulation may include one or more additional sunscreen active agents. Additional sunscreen active agents comprise, but are not limited to, ultraviolet filter compounds, photostabilizers, mineral oxides, or any combination thereof. Exemplary sunscreen active agents include, but are not limited to, avobenzone, cinoxate, dioxybenzone, ensulizole, 2-ethylhexyl 4-(dimethylamino)benzoate (Padimate O), ethylhexyl salicylate, homosalate, meradimate, octinoxate, octocrylene, oxybenzone, polyester-8 (a copolymer of adipic acid and neopentyl glycol end-capped with either octyldodecanol or a cyanodiphenylpropenoyl group), sulisobenzone, titanium dioxide, zinc oxide, or any combination thereof.

In some aspects, the additional sunscreen active agents comprise homosalate, octocrylene, ethylhexyl salicylate, avobenzone, polyester-8, and combinations thereof. In one implementation, the additional sunscreen active agents comprise, consist essentially of, or consist of a combination of homosalate, octocrylene, ethylhexyl salicylate, avobenzone, and polyester-8. The phrase “consist essentially of” in this context means that the sunscreen formulation does not include more than 1 wt % of another sunscreen active agent, based on a total mass of the sunscreen formulation. In one representative example, the additional sunscreen active agents comprise, consist essentially of, or consist of 6 wt % to 8 wt % homosalate, 4 wt % to 6 wt % octocrylene, 2 wt % to 5 wt % ethylhexyl salicylate, 4 wt % to 6 wt % avobenzone, and 1 wt % to 3 wt % polyester-8, where the wt % values are based on a total mass of the sunscreen formulation.

One representative, non-limiting sunscreen stick formulation comprising N-(3-methoxybenzyl)eicos-5-enamide has a composition as shown in the following table:

| Component | Wt % |
|---|---|
| Raphanus sativus (Radish) Seed Extract | 17-26 |
| Limnanthes alba (Meadowfoam) Seed Oil | 16 |
| N-(3-methoxybenzyl)eicos-5-enamide | 1-10 |
| Homosalate | 7 |
| Octocrylene | 5 |
| Ethylhexyl Salicylate/Octisalate | 3 |
| Butyl Methoxydibenzoylmethane/Avobenzone | 5 |
| C12-15 Alkyl Benzoate | 5 |
| Polyester-8 | 2 |
| Bisabolol | 1 |
| Octyldodecanol | 20 |
| Dibutyl Lauroyl Glutamide | 2.4 |
| Dibutyl Ethylhexanoyl Glutamide | 1.6 |

### III. Methods of Making an Amide Lipid Conjugate of a Benzyl Isothiocyanate and a Sunscreen Formulation

In some aspects, making an amide lipid conjugate of a benzyl isothiocyanate (a compound according to Formula I) includes combining a benzyl isothiocyanate and a fatty acid to form a composition; and heating the composition at a temperature of 100° C. to 250° C. for an effective period of time to form the compound according to Formula I, wherein the benzyl isothiocyanate has a structure according to Formula II, where one of R^{1A} and R^{1B} is alkoxy or hydroxy, and the other of R^{1A} and R^{1B} is —H:

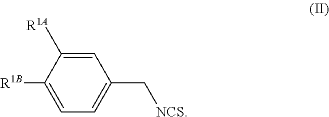

In some aspects, the temperature is within a range having endpoints selected from 100° C., 110° C., 120° C., 130° C., 140° C., 150° C., 160° C., 170° C., 180° C., 190° C., 200° C., 210° C., 220° C., 230° C., 240° C., or 250° C. In certain examples, the temperature is 150° C. to 200° C., such as 160° C. to 170° C. In some implementations, the effective period of time is from 5 minutes to 3 hours. In certain implementations, the effective period of time is within a range having endpoints selected from 5 minutes, 10 minutes, 15 minutes, 30 minutes, 45 minutes, 1 hour, 1.5 hours, 2 hours, 2.5 hours, or 3 hours. In some examples, the effective period of time is from 5 minutes to 60 minutes, such as from 5 minutes to 30 minutes or from 5 minutes to 15 minutes.

In some implementations, the benzyl isothiocyanate is 3-methoxybenzyl isothiocyanate or 4-hydroxybenzyl isothiocyanate. In any of the foregoing or following aspects, the fatty acid may be a C₁₂-C₂₆ fatty acid. In some aspects, the fatty acid is a monounsaturated fatty acid, a polyunsaturated fatty acid, or a saturated fatty acid. In some implementations, the fatty acid is monounsaturated. In certain aspects, the fatty acid is an eicosenoic acid, such as 5-eicosenoic acid. In some examples, the 5-eicosenoic acid is (Z)-5-eicosenoic acid.

In some aspects, making a sunscreen formulation includes heating one or more oil gelling agents at a temperature T₁ effective to form a liquid or solution A; combining a compound according to Formula I, one or more excipients, and, optionally, one or more additional sunscreen active agents and heating at a temperature T₂ effective to form a composition B, wherein composition B is a solution or suspension; and combining the liquid A or solution A and the composition B to provide the sunscreen formulation. When a single oil gelling agent is used, a liquid A is formed when heated at the temperature T₁. When two or more oil gelling agents are used, a solution A is formed when heated at the temperature T₁. In some implementations, the temperature T₁ is 100 to 250° C. In certain implementations, the temperature T₁ is within a range having endpoints selected from 100° C., 105° C., 110° C., 115° C., 120° C., 125° C., 150° C., 175° C., 200° C., 225° C., or 225° C. In some examples, the temperature T₁ is 110° C. to 120° C. In some implementations, the temperature T₂ is 100 to 250° C. In certain implementations, the temperature T₂ is within a range having endpoints selected from 100° C., 105° C., 110° C., 115° C., 120° C., 125° C., 150° C., 175° C., 200° C., 225° C., or 225° C. In some examples, the temperature T₂ is 105° C. to 115° C. In any of the foregoing aspects, the sunscreen formulation may comprise from 0.01 wt % to 15 wt % of the compound according to Formula I. In some aspects, the sunscreen formulation comprises an amount the compound or compounds according to Formula I within a range having endpoints selected from 0.01 wt %, 0.05 wt %, 0.1 wt %, 0.3 wt %, 0.5 wt %, 0.7 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, or 15 wt %. In certain aspects, the sunscreen formulation comprises 0.5 wt % to 15 wt % of the compound(s), such as from 1 wt % to 10 wt % of the compound(s).

### IV. Representative Aspects

Certain representative aspects are exemplified in the following numbered clauses.

1. A compound having a structure according to Formula I:

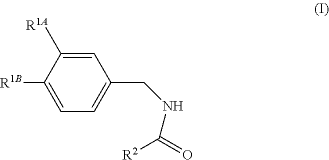

wherein one of R^{1A} and R^{1B} is alkoxy or hydroxy, and the other of R^{1A} and R^{1B} is —H; and R² is C₁₂-C₂₆ aliphatic.

2. The compound of clause 1, wherein one of R^{1A} and R^{1B} is C₁-C₃ alkoxy.

3. The compound of clause 1 or clause 2, wherein R² is unsubstituted C₁₂-C₂₆ alkenyl with one or more double bonds.

4. The compound of clause 1 or clause 2, wherein R² is unsubstituted C₁₈-C₂₂ alkenyl with one or two double bonds.

5. The compound of clause 1, wherein: R^{1A} is methoxy; and R² is —(CH₂)₃CH═CH(CH₂)₁₃CH₃.

6. The compound of clause 1, wherein: R^{1B} is hydroxy; and R² is —(CH₂)₃CH═CH(CH₂)₁₃CH₃.

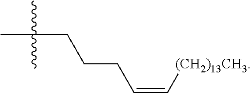

7. The compound of any one of clauses 1-6 where R² is

8. The compound of clause 1, wherein the compound is:

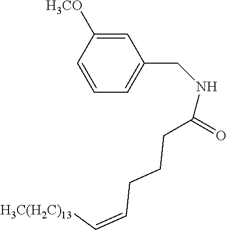

(Z)—N-(3-methoxybenzyl)eicos-5-enamide.

9. The compound of clause 1, wherein the compound is:

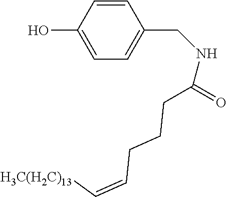

(Z)—N-(4-hydroxybenzyl)eicos-5-enamide.

10. A sunscreen formulation comprising a compound according to any one of clauses 1-9 and one or more excipients.

11. The sunscreen formulation of clause 10, wherein the formulation is a solid, a cream,

a lotion, a gel, an ointment, an oil, or a spray formulation.

12. The sunscreen formulation according to clause 10 or clause 11, wherein the formulation comprises from 0.01 to 15 wt % of the compound.

13. The sunscreen formulation according to any one of clauses 10-12, wherein the excipients comprise a plant or seed extract, a plant or seed oil, an emollient, a humectant, a film-forming agent, a thickener, an emulsifier, a diluent, a fragrance, or any combination thereof.

14. The sunscreen formulation according to any one of clauses 10-13, wherein the excipients comprise a plant or seed extract, a plant or seed oil, an alcohol, an alkyl benzoate, an oil gelling agent, or any combination thereof.

15. The sunscreen formulation according to any one of clauses 10-14, wherein the formulation further comprises one or more additional sunscreen active agents.

16. The sunscreen formulation according to clause 15, wherein the one or more additional sunscreen active agents comprise an ultraviolet filter compound, a photostabilizer, a mineral oxide, or any combination thereof.

17. The sunscreen formulation according to clause 15 or clause 16, wherein the one or more additional sunscreen active agents comprises avobenzone, cinoxate, dioxybenzone, ensulizole, 2-ethylhexyl 4-(dimethylamino)benzoate, ethylhexyl salicylate, homosalate, meradimate, octinoxate, octocrylene, oxybenzone, polyester-8, sulisobenzone, titanium dioxide, zinc oxide, or any combination thereof.

18. The sunscreen formulation according to any one of clauses 10-17, comprising: 0.01 wt % to 15 wt % of the compound; Raphanus spp. seed extract, Sinapis spp. seed extract, or a combination thereof; Limnanthes alba seed oil; homosalate; octocrylene; ethylhexyl salicylate; avobenzone; C₁₂-C₁₅ alkyl benzoate; polyester-8; bisabolol; octyldodecanol; dibutyl lauroyl glutamide; and 1 wt % to 2 wt % dibutylethylhexanoyl glutamide.

19. The sunscreen formulation according to clause 18, comprising: 1 wt % to 10 wt % of the compound; 17 wt % to 26 wt % Raphanus sativus seed extract, Sinapis spp. seed extract, or a combination thereof; 14 wt % to 18 wt % Limnanthes alba seed oil; 6 wt % to 8 wt % homosalate; 4 wt % to 6 wt % octocrylene; 2 wt % to 5 wt % ethylhexyl salicylate; 4 wt % to 6 wt % avobenzone; 4 wt % to 6 wt % C₁₂-C₁₅ alkyl benzoate; 1 wt % to 3 wt % polyester-8; 0.5 wt % to 1.5 wt % bisabolol; 18 wt % to 22 wt % octyldodecanol; 2 wt % to 3 wt % dibutyl lauroyl glutamide; and 1 wt % to 2 wt % dibutylethylhexanoyl glutamide.

20. A method for making a compound according to any one of clauses 1-9, comprising: combining a benzyl isothiocyanate and a fatty acid to form a composition; and heating the composition at a temperature of 100° C. to 250° C. for an effective period of time to form the compound according to Formula I, wherein the benzyl isothiocyanate has a structure according to Formula II, where one of R^{1A} and R^{1B} is alkoxy or hydroxy, and the other of R^{1A} and R^{1B} is —H

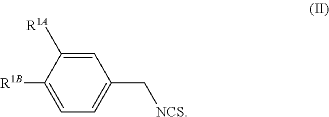

21. The method of clause 20, wherein the effective period of time is from 5 minutes to 3 hours.

22. The method of clause 20 or clause 21 where the benzyl isothiocyanate is 3-methoxybenzyl isothiocyanate or 4-hydroxybenzyl isothiocyanate.

23. The method of any one of clauses 20-22, wherein the fatty acid is a C₁₂-C₂₆ fatty acid.

24. The method of any one of clauses 20-23, wherein the fatty acid is a monounsaturated fatty acid, a polyunsaturated fatty acid, or a saturated fatty acid.

25. The method of clause 24, wherein the fatty acid is an eicosenoic acid.

26. The method of clause 25, wherein the eicosenoic acid is 5-eicosenoic acid.

27. The method of clause 26, wherein the 5-eicosenoic acid is (Z)-5-eicosenoic acid.

28. A method for making a sunscreen formulation, comprising: heating one or more oil gelling agents at a temperature T₁ effective to form a liquid A when there is one oil gelling agent or a solution A when there is more than one oil gelling agent; combining a compound according to any one of clauses 1-9, one or more excipients, and, optionally, one or more additional sunscreen active agents and heating at a temperature T₂ effective to form a composition B, wherein the composition B is a solution or suspension; and combining the liquid A or the solution A and the composition B to provide the sunscreen formulation.

29. The method of clause 28, wherein: (i) the temperature T₁ is 100° C. to 250° C.; or (ii) the temperature T₂ is 100° C. to 250° C.; or (iii) both (i) and (ii).

30. The method of clause 28 or clause 29, wherein the sunscreen formulation comprises from 0.01 wt % to 15 wt % of the compound.

### V. Examples

### Materials and Methods

The following materials and methods may be useful in practicing aspects of the disclosure.

### Synthesis of N-(3-methoxybenzyl)eicos-5-enamide

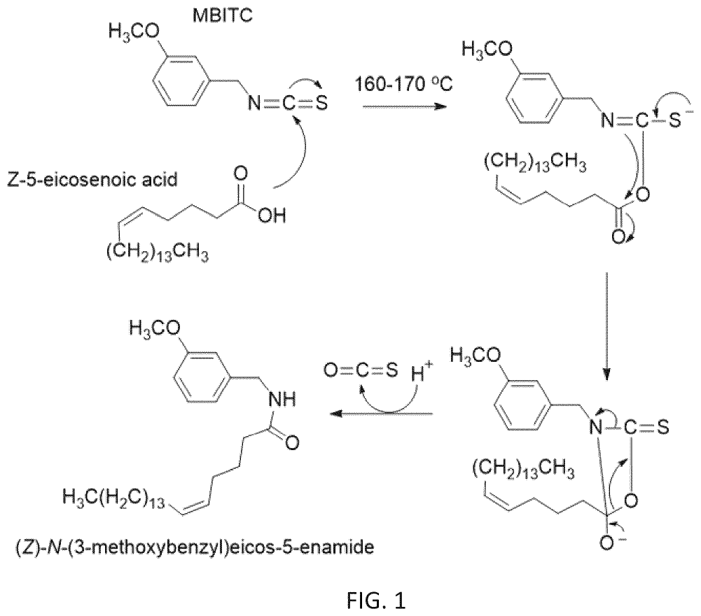

FIG. 1 is a schematic representation of the synthesis of N-(3-methoxybenzyl)eicos-5-enamide (MBamide or MBA) from 3-methoxybenzyl isothiocyanate (MBITC) and the meadowfoam fatty acid, Z-5-eicosenoic acid. Meadowfoam seed oil includes triglycerides whose fatty acids are primarily made up of eicos-5Z-enoic acid (C20: 1; 61%), docos-5Z-enoic acid (C22: 1; 16%), and docosa-5Z,13Z-dienoic acid (C22: 2; 18%). Meadowfoam seed oil (1 mL) was transferred to a 50 mL-round bottom flask, and 5 mL of a solution of KOH (280 mg) in ethanol was added. The mixture was stirred for 3 h at 70° C. After cooling, 3 mL of aqueous 2 N HCl and 12 mL of water were added, and the mixture was extracted three times with 20 mL ethyl acetate. The organic layers were collected, dried over anhydrous Na₂SO₄, filtered, and taken to dryness by rotary evaporation. An aliquot of the hydrolyzed oil (150 μL) and 60 μL of MBITC (Oakwood Products, West Columbia, SC, USA) were transferred to a 2-mL glass vial, and the contents vortexed for 20 s. The vial was capped, and the contents heated for 6 h at 160° C. After cooling, the mixture was diluted with 50 mL ethyl acetate and washed with saturated aqueous NaHCO₃ (3×50 mL) and water (2×50 mL). The organic layer was dried over anhydrous Na₂SO₄, filtered, taken to dryness by rotary evaporation, and the residue reconstituted in 1 mL of ethyl acetate. The product was purified by open-column silica gel chromatography using hexane-ethyl acetate (5:1, 300 mL, 3:1, 400 mL; 1:1, 300 mL) and ethyl acetate (300 mL) as solvents. Fractions were collected and monitored by silica gel TLC developed in dichloromethane. The amide-containing fractions were combined and evaporated, and the product analyzed by LC-MS/MS. The major product was consistent with the molecular formula (C₂₈H₄₇NO₂, Δ=−3.4 ppm) and with the production of a methoxybenzyl fragment ion observed at m/z 121.

### UVB Absorption Assay

MBITC or MBA was diluted in ethanol at 0, 1, 10, 25, and 50 μM, added to separate wells of a 24-well plate (Corning, Corning, NY, USA), and irradiated with UVB light using a UV Transilluminator 2000 (Bio-Rad, Hercules, CA, USA) to reach 5 mJ/cm² or 10 mJ/cm². A Daavlin X-96 irradiance meter (Daavlin, Bryan, OH, USA) measured energy transmittance through the dish. Each well was separately irradiated, and the time was measured to reach the specified energy density.

### Cell Culture

Immortalized keratinocyte HaCaT cells (ATCC, Manassas, VA, USA) were cultured in DMEM supplemented with 5% fetal bovine serum. Human primary epidermal keratinocytes (HPEKs) (ATCC) were cultured in EpiLife medium (ThermoFisher Scientific) supplemented with Human Keratinocyte Growth Supplement (ThermoFisher Scientific). HPEKs were maintained below ten passages. Cell lines were incubated in a humidified environment at 37° C. with 5% CO₂.

### Dose-Response and Time Kinetics Assays

For viability studies, HaCaT cells were seeded 5000 cells/well in clear 96-well plates (Corning, Corning, NY, USA). After 24 h, cells were treated with MBITC or MBA at 0, 3.125, 6.25, 12.5, 25, or 50 μM and incubated with the compounds for 48 h. Viability was then determined by MTS assay using CellTiter 96® AQueous One Solution Cell Proliferation Assay (Promega Corporation, Madison, WI, USA). Absorption was measured using Cytation® 5.0 Imaging Reader (Agilent, Santa Clara, CA, USA). HPEKs were seeded 5000 cells/well into white, opaque bottom 96-well plates (Greiner Bio-One) and incubated for 48 h. HPEKs were then treated with ethanol vehicle control or a 5-point half-log serial dilution of MBA starting at 50 μM. After 48 h, CellTiter-Glo® (Promega Corporation, Madison, WI) viability assays were performed per the manufacturer's instructions, and luminescence was read using a Synergy4 (Biotek, Winooski, VT) plate reader.

For UV protection dose-response assays, HaCaT cells were seeded 5000 cells/well into clear 96-well plates (Corning). After 24 h, cells were treated with MBITC or MBA at 0, 3.125, 6.25, 12.5, 25, or 50 UM and incubated for 12 h. Cells were washed in PBS and irradiated with 10 mJ/cm² UVB using a UV Transilluminator 2000. After irradiation, fresh media was added, cells incubated for 36 h, and viability was determined by MTS assay as before. HPEKs were seeded 5000 cells/well into black, opaque bottom 96-well plates (Greiner Bio-One, Monroe, NC, USA) and incubated for 48 h. Afterward, cells were treated with vehicle control or a 10-point half-log serial dilution of MBITC or MBA starting at 50 μM and incubated for 12 h. Before irradiation, the conditioned cell culture medium was re-moved from each well, retained, and PBS containing the same dose of EtOH or the compounds was added to the wells. Cells were irradiated with a 10 mJ/cm² dose using UVB TL 20W/12 RS M3 lights (Philips, Amsterdam, Netherlands). The dose of UVB was quantified using an IL1400A NIST Traceable Photometer (International Light, Peabody, MA, USA). Following irradiation, the PBS was aspirated from the wells, and conditioned media was added. After 36 h, CYQUANT® (ThermoFisher Scientific) assays were performed per the manufacturer's instructions, and fluorescent signal read using a Synergy4® (Biotek, Winooski, VT) plate reader. Cell counts were extrapolated from a standard curve of fluorescent signal activity vs. HPEK cell counts. Time kinetics involved the same compound and UVB treatment scheme as before, but CYQUANT® assays were performed at 24, 48, 72, or 96 h post-UVB irradiation.

### Immunocytochemistry

HPEKs were cultured to 80% confluence in 6-well plates containing gelatin-coated coverslips and treated with ethanol vehicle control or MBA. After 12 h, the conditioned cell culture medium was removed, retained, and PBS containing EtOH or MBA was added to each well. Plates were then irradiated with 5 mJ/cm² UVB, PBS removed, conditioned media re-added, and incubated for 24 h. Cells were then fixed in 75% methanol/25% acetic acid, washed with 70% EtOH, and DNA denatured in 70 mM NaOH (in 70% EtOH). Wells were then washed 3 times with PBS and denaturation quenched with 100 mM Tris —HCl (in 70% EtOH). Cells were then blocked with 10% normal goat serum (NGS) in PBS-T and incubated overnight at 4° C. with α-thymine dimer primary antibody (1:1000, Kamiya Bio-medical, Matsumoto, Nagano, Japan) in NGS blocking buffer. The next day, incubated with Cy3 goat α-mouse secondary antibody (1:50, Jackson Immunolabs, West Grove, PA, USA) in PBS-T. Cells were counterstained with 0.2 ng/mL DAPI, dehydrated through an ethanol gradient, and coverslips mounted to slides using dibutyl phthalate in xylene (VWR, Radnor, PA, USA). Fluorescent microscopy used a Zeiss AXIO Imager.Z1 (Carl Zeiss, Oberkochen, Germany). For each independent experiment, treatments were performed in duplicate, and 10 fields were imaged per replicate. Counts of CPD+ cells were performed by two investigators blinded from experimental groups of the samples. Corrected total cellular fluorescence (CTCF) was quantified using the ImageJ software package as described previously, except only nuclear CTCF for CPD staining was quantified and nuclei were selected for ROIs using manual thresholding for each field (Carpenter et al., J Invest Dermatol 2022, 142:1903-1911). For all quantifications, cells on the edges of each field were excluded.

### Immunoblotting

HPEKs were cultured to 80% confluence in 6-well plates and treated with ethanol vehicle control or MBA. Cells were then irradiated in the same manner as immunocytochemistry samples. After 36 h, cells were harvested by scraping into cold PBS, centrifuged, pellet resuspended in denaturing SDS lysis buffer, boiled, and sonicated as described previously (Carpenter et al., Mol Carcinog 2019, 58:1680-1690). Samples were normalized to protein content as determined by BCA Assay (ThermoFisher Scientific). SDS-PAGE was then performed using 10% gels and transferred to a 0.45 μm nitrocellulose membrane (GE Healthcare, Chicago, IL, USA). Blots were blocked for 1 h in 5% nonfat dry milk in TBS-T and incubated with α-p-γH₂A.X antibody (1:1000, Cell Signaling Technology, Danvers, MA, USA) or Actin (1:2000, Bethyl Laboratories, Inc., Montgomery, TX, USA) in 5% bovine serum albumin in TBS-T overnight at 4° C. The next day, blots were incubated with HRP-conjugated Goat anti-rabbit IgG or Goat anti-mouse IgG secondary antibodies (1:2000, Calbiochem, San Diego, CA, USA) for 1 h. The immunoblots were developed using chemiluminescent reagents (GE Healthcare) and imaged with a MyECL imager (ThermoFisher Scientific).

### Software and Statistics

The imaging software used for fluorescence microscopy was Axio Vision 4.8 (Carl Zeiss), and fluorescence images were processed using Photoshop (Adobe, San Jose, CA, USA). CTCF and immunoblotting were quantified using ImageJ 1.54f (National Institutes of Health, Bethesda, MD, USA). Statistics were performed with Prism 10 (GraphPad Software, San Diego, CA, USA). Significance was determined either by one-way ANOVA or a one-sample t-test.

### Example 1. Reaction of MBITC with Meadowfoam Fatty Acids Forms N-(3-Methoxybenzyl)Eicos-5-Enamide (MBA) and Related Fatty Acid-Derived Amides

Meadowfoam oil was saponified, and the resulting mixture of meadowfoam fatty acids (MFAs) was isolated. Using established procedures reported in the literature (FIG. 1), MBITC was mixed with MFAs in equimolar amounts, and the mixture was heated at 160-170° C. for periods of up to 15 minutes with magnetic stirring. The formation of MBITC-MFA amide conjugates was monitored by LC-MS/MS. After optimization of the yield under various conditions, conjugates were produced at a larger scale (0.1-1 g), the products were purified by column chromatography to remove residual MBITC and MFAs, and the mixture of MBITC-MFA conjugates, or MBA, was then tested for biological activity.

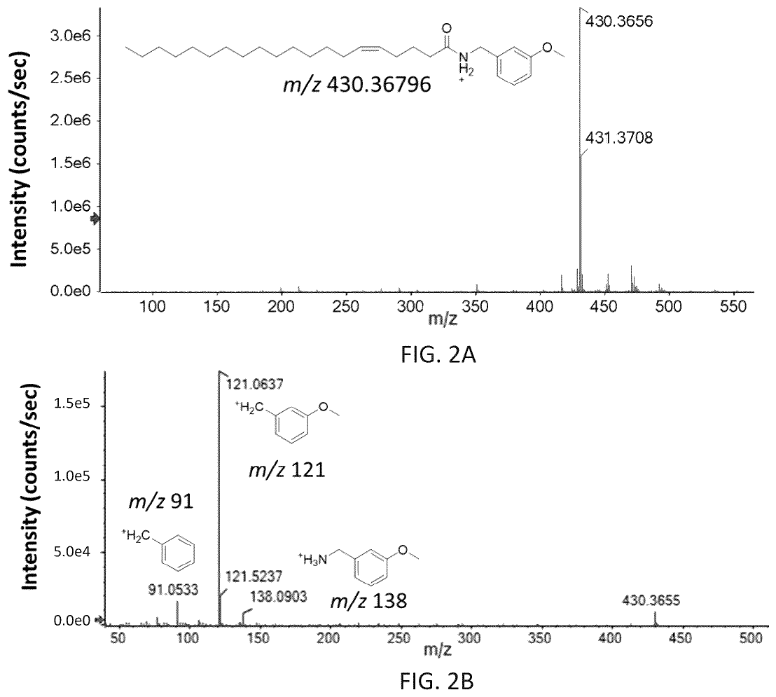

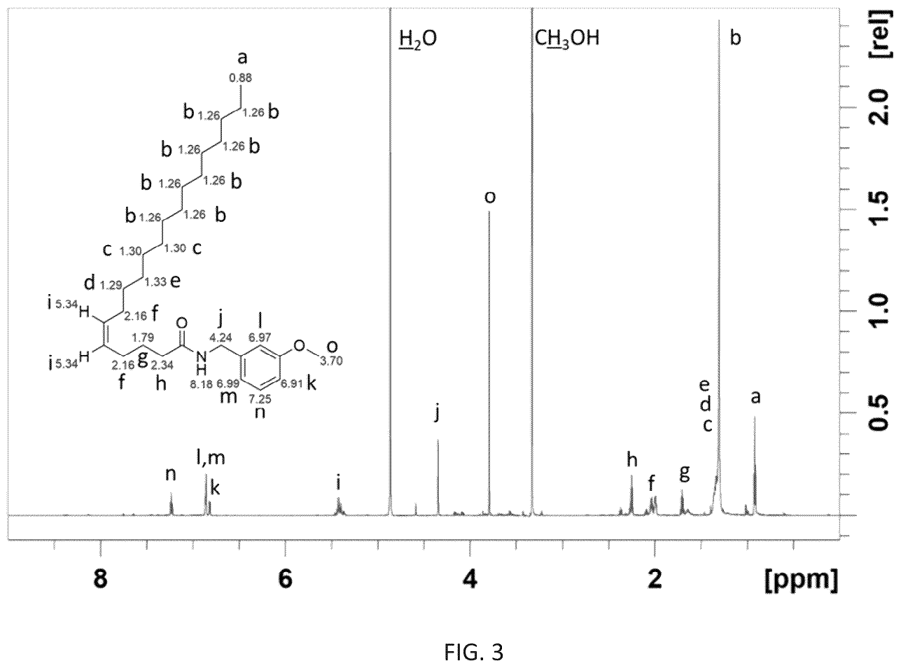

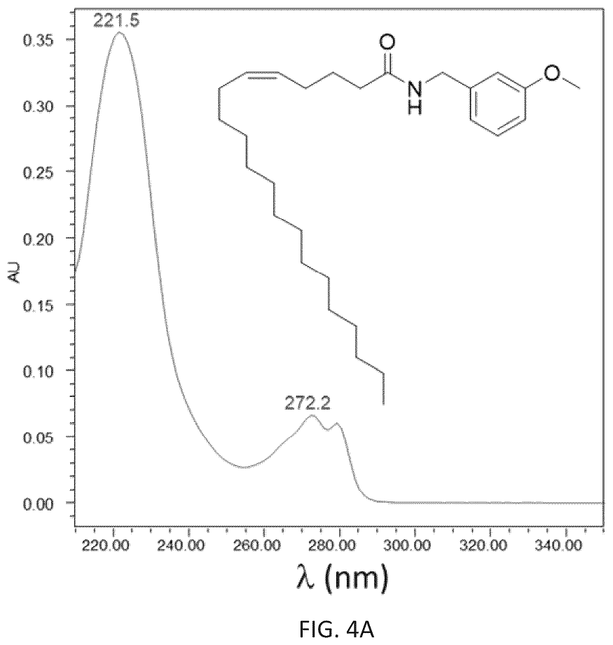

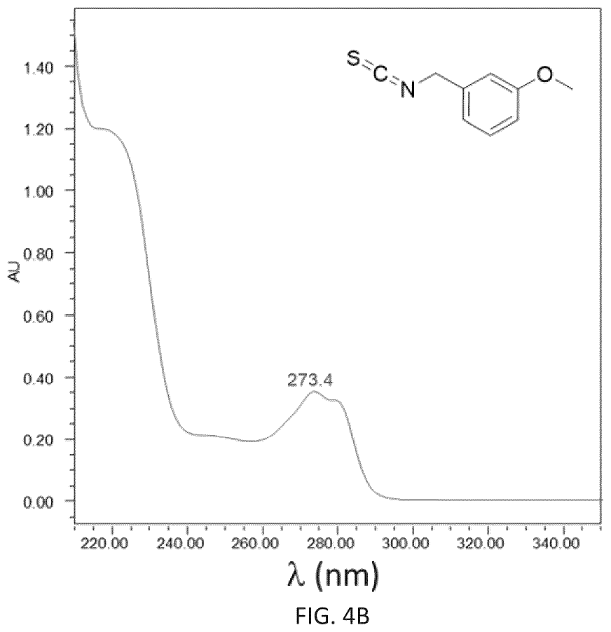

Time-of-Flight (ToF) mass spectroscopy of (Z)—N-(3-methoxybenzyl)eicos-5-enamide, prepared from 3-methoxybenzyl isothiocyanate and meadowfoam-derived fatty acids provided an observed m/z 430.3656; calculated for [C₂₈H₄₇NO₂]⁺: m/z 430.36796; mass error-5.4 ppm (FIG. 2A). A Q-ToF MS/MS spectrum of the molecular ion shows expected mass fragments with m/z 138, 121, and 91 (FIG. 2B). FIG. 3 shows the 1H NMR Spectrum of (Z)—N-(3-methoxybenzyl)eicos-5-enamide (MBamide) in methanol-d4 (700 MHz). The structure of MBamide shows predicted chemical shifts (ppm) and annotated protons. UV spectra of (Z)—N-(3-methoxybenzyl)eicos-5-enamide (MBamide) (FIG. 4A) and 3-methoxybenzyl isothiocyanate (MBITC) (FIG. 4B), show that the benzyl chromophore remains intact after acyl conjugation of MBITC (λₘₐₓ 273 nm)

### Example 2. MBA Exhibits Reduced Cytotoxicity Compared to MBITC

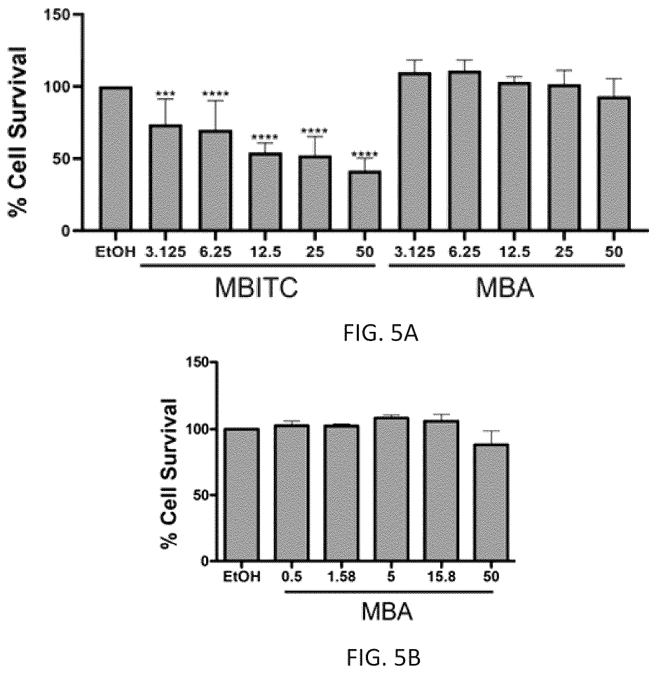

To examine the effect of lipid conjugation on the compound's cytotoxicity, dose-response viability assays were performed with MBA and MBITC in the absence of UVB irradiation. A previous study observed that MBITC exhibited cytotoxic effects in HPEKs under these conditions. In immortalized HaCaT cells, MBITC exhibited significant cytotoxicity at all the tested doses, whereas MBA had little to no effect (FIG. 5A). In HPEKs, MBA was also not cytotoxic at the tested doses (FIG. 5B). Overall, these data confirmed the previous findings regarding MBITC and showed that lipid conjugation of the parental structure reduced this deleterious side effect.

### Example 3. MBA and MBITC Absorb UVB Light but do not Prevent a UVB-Induced Loss in Proliferation in 2D Keratinocyte Cultures

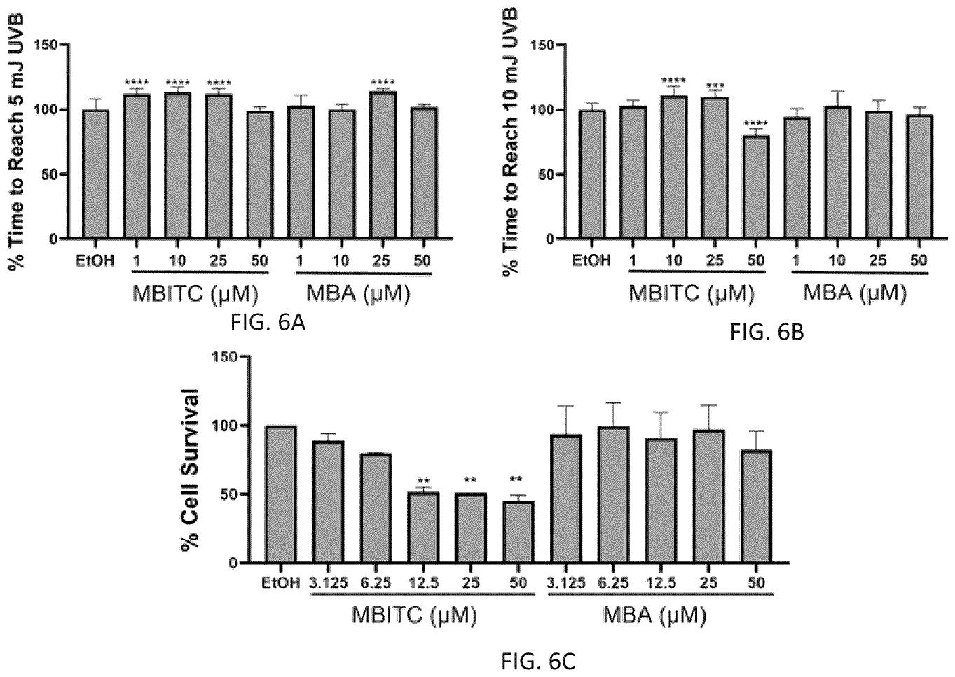

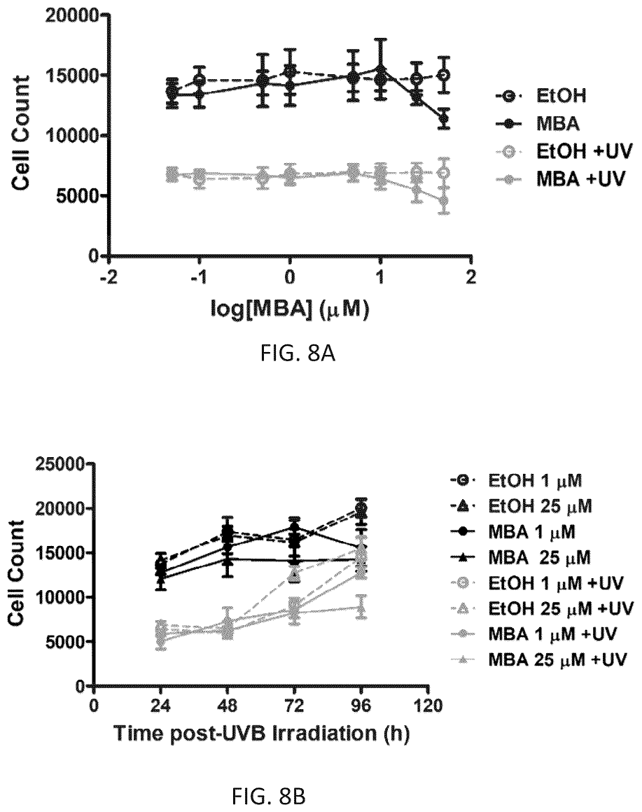

UVB absorption assays were performed for both MBITC and MBA to determine if the structure's photoprotective properties were maintained following lipid conjugation. The amount of UVB light absorbed by MBITC was sufficient to increase the time needed to reach a 5 mJ/cm² dose at every tested concentration except 50 μM, where the time significantly decreased. On the other hand, MBA only showed similar activity at a single concentration of 25 μM (FIG. 6A). A similar trend was also seen in the time needed to reach 10 mJ/cm². MBITC exhibited more UVB absorption capacity than MBA, suggesting lipid conjugation interferes with the structure's absorptive capacity (FIG. 6B). The ability of both compounds to protect HaCaT cells from a UVB-induced loss in proliferation was then evaluated. Similar to a previous study, MBITC did not reverse the loss of proliferation following irradiation with 10 mJ/cm² UVB and displayed cytotoxic effects in the presence of UVB light (FIG. 6C). Interestingly, MBA did not exhibit the same cytotoxic effects as the parental compound at the tested concentrations. The capability of MBA to prevent a UV-induced loss in proliferation in human primary keratinocytes (HPEKs) was also examined, where the compound had the same effect as in HaCaT cells (FIG. 8A). Even increasing the incubation time to four days did not reveal a protective effect of MBA under these conditions (FIG. 8B).

### MBA Reduces UVB-Induced DNA Damage

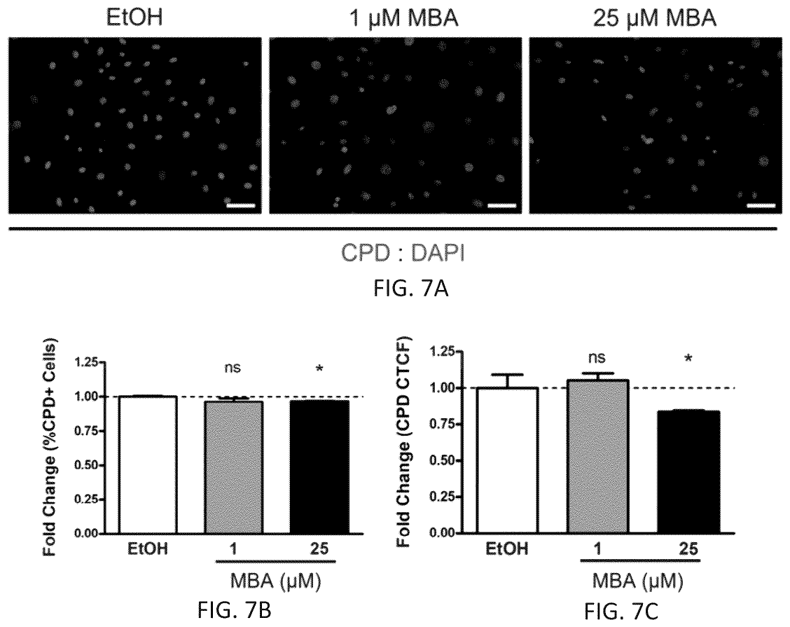

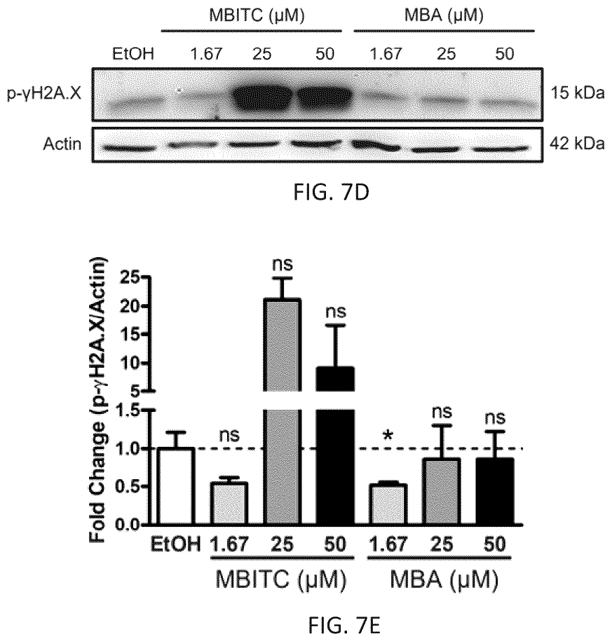

In a previous study, the parental compound MBITC could reduce UVB-induced DNA damage in the form of cyclobutane pyrimidine dimers (CDPs) and p-γH2A.X, a marker for DNA double-stranded breaks. To determine whether the lipid-conjugated structure could engage in the same activity, the effect of MBA treatment on cyclobutane pyrimidine dimer (CPD) formation after 5 mJ/cm² UVB irradiation of HPEKs was evaluated (FIG. 7A). This lower UVB dose was chosen based on the absorption assay results, which indicated a protective effect of MBA treatment might be observed under these conditions. A small but significant difference in the total number of CPD+ cells between MBA treatment and vehicle control was found (FIG. 7B). However, treatment with MBA substantially decreased the intensity of the CPD staining in irradiated HPEKs (FIG. 7C). These data indicate that MBA can prevent UVB-induced formation of CPDs, but likely to a lesser degree than MBITC, where the previous work demonstrated that MBITC was more potent in preventing CPD formation. Immunoblotting for p-γH2A.X was performed in HPEKs irradiated with the same dose of UVB (FIG. 7D). Surprisingly, MBITC exhibited a trend of increased p-γH2A.X staining compared to MBA at 25 and 50 μM concentrations and showed less reduction at 1 μM (FIG. 7E).

Previous work found that the meadowfoam glucosinolate derivative MBITC had photoprotective properties by reducing UVB-induced DNA damage, proliferation, and matrix metalloproteinase expression (Carpenter et al., Front Pharmacol 2018, 9:477). However, the compound exhibited significant cytotoxic effects at higher concentrations. Therefore, compounds modifications were sought to mitigate these side effects and improve it as a potential sunscreen additive.

Electrophilic compounds, like MBITC, can react with various biomolecules, including proteins and DNA, causing accumulated damage that is ultimately cytotoxic (LoPachin et al., Free Radic Res 2016, 50:195-205). Alternatively, UV-absorptive molecules, like the photoprotective pigment melanin, can have damaging effects following excitation. In this excited state, energy can then be transferred to DNA, causing CPDs (Premi et al., Science 2015, 347-842-847). Reactivity of electrophilic MBITC with biomolecules or UVB-induced photosensitization could be potential mechanisms by which MBITC exerts its cytotoxic effects. Conjugation of MBITC with meadowfoam fatty acids potentially could alter the subcellular localization of the compound such that MBA would more readily associate with lipid membranes rather than reacting with cytosolic proteins or nuclear DNA.

The study demonstrated that lipid conjugation reduced the cytotoxicity of a photoprotective natural product derivative. This is seen by the increased viability of keratinocytes treated with equivalent doses of MBA compared to MBITC in the presence or absence of UVB. Other studies have found that lipid-drug conjugates synthesized using an amide bond reduce the cytotoxicity of anti-cancer compounds like doxorubicin (Duhem et al., Bioconjug Chem 2014, 25:72-81). The reason for this is that hydrolysis of the amide bond is necessary for the drug to be released and exert its anti-cancer activity. Based on the absorbance assay, it is clear that MBA does not require hydrolysis for its function.

After treatment with MBA, a lack of elevated p-γH2A.X was seen at higher concentrations following UV irradiation compared to the parental compound. Interestingly, this induction of p-γH2A.X by MBITC was not seen in a previous study using human skin equivalents. This is likely because the effect is dose-dependent, and the dose per keratinocyte likely is drastically reduced in reconstructed skin compared to a 2D monolayer culture given the same treatment concentration. As mentioned earlier, MBITC could elevate p-γH2A.X directly through electrophilic attack, photosensitization after UV absorption, or a combination of both. Lipid conjugation of the structure seems to substantially reduce this activity, which could partially explain the reduced cytotoxicity that was observed. Without wishing to be bound by a particular theory of operation, one explanation could be reduced interaction between MBA and DNA as it preferentially associates with lipid membranes. DNA strand breaks due to photosensitization have been seen with other UV-absorbing sunscreen additives such as 2-phenylbenzimidazole-5-sulfonic acid (PBSA) (Bastien et al., J Invest Dermatol 2010, 130:2463-2471).

The UV absorptive capacity of MBITC appeared to be affected by lipid conjugation, as a significant degree of absorption was only observed at a lower dose of 5 mJ/cm² UVB. However, MBA still reduces DNA damage following irradiation, as seen in our immunohistochemistry staining for CPDs and immunoblotting for p-γH2A.X. This activity of MBA may be attributable to its absorption of UVB preventing its incidence on the DNA of cell cultures. MBA slightly reduced the number of keratinocytes with no CPD staining but more substantially reduced the CPD signal per cell. Compared to previous work with MBITC, MBA was less potent towards reducing CPDs, most likely due to its reduced UV absorbency. Interestingly, there was a trend of reduced p-γH2A.X only at the lowest tested dose of MBA and MBITC, and the reduced effect was lost at higher doses. As mentioned earlier for MBITC, the trend of increased p-γH2A.X appears correlated with its cytotoxicity. For MBA, it could be that some compound can still interact with DNA, possibly due to hydrolysis of the amide bond releasing the parental compound.

The results show, for the first time to the inventors' knowledge, that lipid conjugation reduces a photoprotective natural product's cytotoxicity while retaining its UVB absorptive capability. The compound can be further evaluated in a skin-like environment, such as reconstructed skin, and in different formulations, like in a solid lipid nanoparticle.

Sunscreen formulations include one or more active ingredients and a vehicle medium, allowing for effective dosing and coverage of the skin. MBA can either be the sole active ingredient or used in combination with other actives used in current commercial sunscreen products. MBA is ethanol soluble, making it capable of being added to oil-based, ethanol/oil-based, or water-oil emulsion-based sunscreen formulations (Tanner et al., Dermatol Clin 2006, 24 (1): 53-63). Examples of oil-based sunscreens include sticks, ointments, oily gels, oils, and sprays. Ethanol/oil-based sunscreens include gels and sprays, and water-oil emulsion-based sunscreens include creams and lotions.

We claim:

1. A compound having a structure according to Formula I: 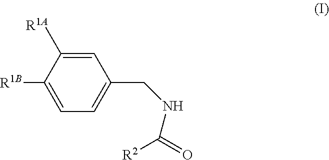
wherein one of R^{1A} and R^{1B} is alkoxy or hydroxy, and the other of R^{1A} and R^{1B} is —H; and
R² is C₁₂-C₂₆ aliphatic.

2. The compound of claim 1, wherein:
one of R^{1A} and R^{1B} is C₁-C₃ alkoxy; or
R^{1B} is hydroxy.

3. The compound of claim 1, wherein R² is unsubstituted C₁₂-C₂₆ alkenyl with one or more double bonds.

4. The compound of claim 1 where R² is

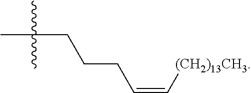

5. The compound of claim 1, wherein the compound is: 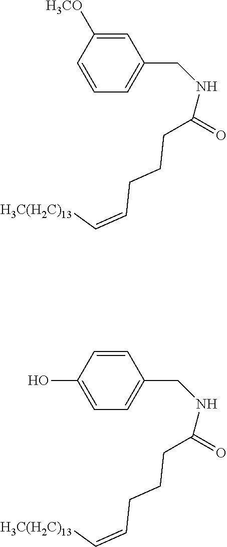
(Z)—N-(3-methoxybenzyl)eicos-5-enamide or (Z)—N-(4-hydroxybenzyl)eicos-5-enamide.

6. A sunscreen formulation comprising a compound according to claim 1 and one or more excipients.

7. The sunscreen formulation of claim 6, wherein the formulation is a solid, a cream, a lotion, a gel, an ointment, an oil, or a spray formulation.

8. The sunscreen formulation according to claim 6, wherein the formulation comprises from 0.01 to 15 wt % of the compound.

9. The sunscreen formulation according to claim 6, wherein the excipients comprise a plant or seed extract, a plant or seed oil, an emollient, a humectant, a film-forming agent, a thickener, an emulsifier, a diluent, a fragrance, or any combination thereof.

10. The sunscreen formulation according to claim 6, wherein the excipients comprise a plant or seed extract, a plant or seed oil, an alcohol, an alkyl benzoate, an oil gelling agent, or any combination thereof.

11. The sunscreen formulation according to claim 6, wherein the formulation further comprises one or more additional sunscreen active agents selected from an ultraviolet filter compound, a photostabilizer, a mineral oxide, or any combination thereof.

12. The sunscreen formulation according to claim 11, wherein the one or more additional sunscreen active agents comprises avobenzone, cinoxate, dioxybenzone, ensulizole, 2-ethylhexyl 4-(dimethylamino) benzoate, ethylhexyl salicylate, homosalate, meradimate, octinoxate, octocrylene, oxybenzone, polyester-8, sulisobenzone, titanium dioxide, zinc oxide, or any combination thereof.

13. The sunscreen formulation according to claim 6, comprising:
0.01 wt % to 15 wt % of the compound;
Raphanus spp. seed extract, Sinapis spp. seed extract, or a combination thereof;
Limnanthes alba seed oil;
homosalate;
octocrylene;
ethylhexyl salicylate;
avobenzone;
C₁₂-C₁₅ alkyl benzoate;
polyester-8;
bisabolol;
octyldodecanol;
dibutyl lauroyl glutamide; and
1 wt % to 2 wt % dibutylethylhexanoyl glutamide.

14. The sunscreen formulation according to claim 13, comprising:
1 wt % to 10 wt % of the compound;
17 wt % to 26 wt % Raphanus sativus seed extract, Sinapis spp. seed extract, or a combination thereof;
14 wt % to 18 wt % Limnanthes alba seed oil;
6 wt % to 8 wt % homosalate;
4 wt % to 6 wt % octocrylene;
2 wt % to 5 wt % ethylhexyl salicylate;
4 wt % to 6 wt % avobenzone;
4 wt % to 6 wt % C₁₂-C₁₅ alkyl benzoate;
1 wt % to 3 wt % polyester-8;
0.5 wt % to 1.5 wt % bisabolol;
18 wt % to 22 wt % octyldodecanol;
2 wt % to 3 wt % dibutyl lauroyl glutamide; and
1 wt % to 2 wt % dibutylethylhexanoyl glutamide.

15. A method for making a compound according to claim 1, comprising:
combining a benzyl isothiocyanate and a fatty acid to form a composition; and
heating the composition at a temperature of 100° C. to 250° C. for an effective period of time to form the compound according to Formula I, wherein the benzyl isothiocyanate has a structure according to Formula II, where one of R^{1A} and R^{1B} is alkoxy or hydroxy, and the other of R^{1A} and R^{1B} is —H

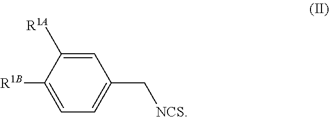

16. The method of claim 15, wherein
(i) the benzyl isothiocyanate is 3-methoxybenzyl isothiocyanate or 4-hydroxybenzyl isothiocyanate; or
(ii) the fatty acid is a C₁₂-C₂₆ fatty acid; or
(iii) both (i) and (ii).

17. The method of claim 15, wherein the fatty acid is an eicosenoic acid.

18. A method for making a sunscreen formulation, comprising:
heating one or more oil gelling agents at a temperature T₁ effective to form a liquid A when there is one oil gelling agent or a solution A when there is more than one oil gelling agent;
combining a compound according to claim 1, one or more excipients, and, optionally, one or more additional sunscreen active agents and heating at a temperature T₂ effective to form a composition B, wherein the composition B is a solution or suspension; and
combining the liquid A or the solution A and the composition B to provide the sunscreen formulation.

19. The method of claim 18, wherein:
(i) the temperature T₁ is 100° C. to 250° C.; or
(ii) the temperature T₂ is 100° C. to 250° C.; or
(iii) both (i) and (ii).

20. The method of claim 18, wherein the sunscreen formulation comprises from 0.01 wt % to 15 wt % of the compound.
